(19) United States
(12) Patent Application Publication
(10) Pub. No.: US 2019/0275233 A1
(43) Pub. Date: Sep. 12, 2019

(54) DRIP CYLINDER NOZZLE AND DRIP CYLINDER

(71) Applicant: Murata Manufacturing Co., Ltd., Kyoto (JP)

(72) Inventors: Takahiro YAMAGUCHI, Kyoto (JP); Koji MIYABAYASHI, Kyoto (JP); Kazuma KAWAI, Kyoto (JP)

(21) Appl. No.: 16/420,355

(22) Filed: May 23, 2019

Related U.S. Application Data
(63) Continuation of application No. PCT/JP2017/042012, filed on Nov. 22, 2017.

(30) Foreign Application Priority Data
Nov. 30, 2016 (JP) 2016-232582

(51) Int. Cl. A61M 5/14; B05B 1/30; B05B 12/08

(52) U.S. Cl. CPC A61M 5/14; A01G 25/023; B05B 12/081; B05B 1/30

(57) ABSTRACT

A dripping cylinder includes a housing, a nozzle that has an inflow hole which allows a liquid to flow into the housing and that causes the liquid to drip from an end portion of the inflow hole, and a needle-shaped portion that is held in a state in which at least an end portion of the needle-shaped portion is positioned further downstream than the end portion of the inflow hole of the nozzle in a drip direction.

This is a continuation of International Application No. PCT/JP2017/042012 filed on Nov. 22, 2017 which claims priority from Japanese Patent Application No. 2016-232582 filed on Nov. 30, 2016. The contents of these applications are incorporated herein by reference in their entireties.

### BACKGROUND

### Technical Field

The present disclosure relates to a drip cylinder nozzle and a drip cylinder.

In the related art, there is known a drip cylinder including a cylindrical housing and a nozzle that causes a liquid to drip into the housing (see, for example, Patent Document 1).

The nozzle of the drip cylinder has an inflow hole (corresponding to a lumen in Patent Document 1) into which a liquid can flow. The inflow hole causes the liquid to drip from an end thereof that faces the interior of the housing.
Patent Document 1: Japanese Unexamined Patent Application Publication No. 2003-260135

### BRIEF SUMMARY

In a drip cylinder such as that described above, when the magnitude of gravity applied to a liquid that has collected in an end portion of an inflow hole, which is an end of a nozzle, that is, the weight of the liquid, becomes larger than a force in the antigravity direction that is generated due to surface tension, part of the liquid is separated from the end of the nozzle and drips as liquid droplets. In this case, there is a possibility that the liquid droplets will separate into main droplets and satellite droplets which are smaller than the main droplets. For example, when such satellite droplets are generated, there is a possibility that the satellite droplets will adhere to the inner wall of the housing as a result of, for example, bouncing back from the liquid surface of the liquid that has collected in the housing, so that the visibility of the interior of the housing will deteriorate due to the adhered satellite droplets.

The present disclosure has been made in view of the above-described problem, and the present disclosure provides a drip cylinder capable of suppressing generation of satellite droplets.

A dripping cylinder that solves the above-described problem includes a housing, a nozzle that has an inflow hole which allows a liquid to flow into the housing and that causes the liquid to drip from an end of the inflow hole, and a needle-shaped portion that is held in a state in which at least an end portion of the needle-shaped portion is positioned further downstream than the end of the inflow hole of the nozzle in a drip direction.

With this configuration, the end portion of the needle-shaped portion is positioned further downstream than the end of the inflow hole of the nozzle in the drip direction. Here, in a nozzle that has a configuration of the related art, when a liquid that has collected in an end portion of an inflow hole of the nozzle is separated from the end portion of the inflow hole, the liquid tries to return into the nozzle (the inflow hole) by the surface tension of the liquid on the nozzle (the inflow hole) side. However, part of the liquid cannot completely return into the nozzle (the inflow hole) and separates as satellite droplets from the end portion of the inflow hole. In contrast, in the drip cylinder of the present disclosure, as described above, the needle-shaped portion is positioned further downstream in the drip direction than the end of the inflow hole of the nozzle where satellite droplets are likely to be generated, so that the liquid may easily return into the nozzle through the needle-shaped portion by the surface tension of the liquid. Therefore, generation of the satellite droplets can be suppressed.

In the above-described drip cylinder, the needle-shaped portion can be held in a state of being spaced apart from the inflow hole. The needle-shaped portion that is spaced apart from the inflow hole (e.g., the end of the inflow hole) contributes to further reduction of the satellite droplets.

In the above-described drip cylinder, the needle-shaped portion can be disposed at the center of the inflow hole when an opening surface of the inflow hole is viewed from the front (viewed in a direction perpendicular to an extending direction of the needle-shaped portion).

With this configuration, as a result of the needle-shaped portion being disposed at the center of the inflow hole when the opening surface of the inflow hole is viewed from the front, the liquid that has collected in the end of the inflow hole and the needle-shaped portion are likely to come into contact with each other with higher certainty, and thus, the liquid may easily return into the nozzle with higher certainty through the needle-shaped portion by the surface tension of the liquid after main droplets have dripped.

In the above-described drip cylinder, the needle-shaped portion can include a columnar portion and an end portion.

With this configuration, the needle-shaped portion can be formed of the columnar portion and the end portion so as to have a tapered shape.

In the above-described drip cylinder, the needle-shaped portion can include a cylindrical columnar portion and that the nozzle can have a circular inflow hole. In addition, the ratio of a diameter D1 of the cylindrical columnar portion to a diameter D2 of the circular inflow hole can be set such that D1:D2 is within a range of 1:20 to 3:4.

With this configuration, the ratio of a diameter D1 of the cylindrical columnar portion to a diameter D2 of the circular inflow hole is set such that D1:D2 is within a range of 1:20 to 3:4, so that the flow rate of the liquid that is supplied from the nozzle can be stabilized while generation of satellite droplets is suppressed.

A drip cylinder according to the present disclosure provides an advantageous effect in which generation of satellite droplets can be suppressed.

### BRIEF DESCRIPTION OF THE SEVERAL VIEWS OF THE DRAWINGS

FIG. 1 is a schematic diagram illustrating a configuration of a drip cylinder according to an embodiment of the present disclosure.

FIG. 2 is a perspective view of a cap of the drip cylinder according to the embodiment.

FIG. 3 is a plan view of the cap of the drip cylinder according to the embodiment.

FIG. 4 is a sectional view of a nozzle taken along line 4-4 of FIG. 3.

FIG. 5 is a sectional view of a nozzle according to a modification.

FIG. 6 is a sectional view of a nozzle according to another modification.

FIG. 7 is a perspective view of a needle-shaped portion according to another modification.

FIG. 8 is a perspective view of a needle-shaped portion according to another modification.

FIG. 9 is a perspective view of a needle-shaped portion according to another modification.

FIGS. 10A and 10B are each a sectional view of a nozzle according to another modification.

FIG. 11A is a perspective view of a needle-shaped portion according to another modification, and FIG. 11B is a schematic sectional view of a cap in which the needle-shaped portion illustrated in FIG. 11A is incorporated.

FIGS. 12A and 12B are each a schematic sectional view of a needle-shaped portion according to another modification.

FIG. 13 is a schematic diagram illustrating a needle-shaped portion according to another modification.

FIG. 14 is a schematic diagram illustrating a needle-shaped portion according to another modification.

FIG. 15 is a schematic diagram illustrating a needle-shaped portion according to another modification.

FIG. 16 is a perspective view of a needle-shaped portion according to another modification.

### DETAILED DESCRIPTION

A drip cylinder according to an embodiment of the present disclosure will be described below with reference to the accompanying drawings. Note that some components are illustrated in an enlarged manner in the accompanying drawings for ease of understanding.

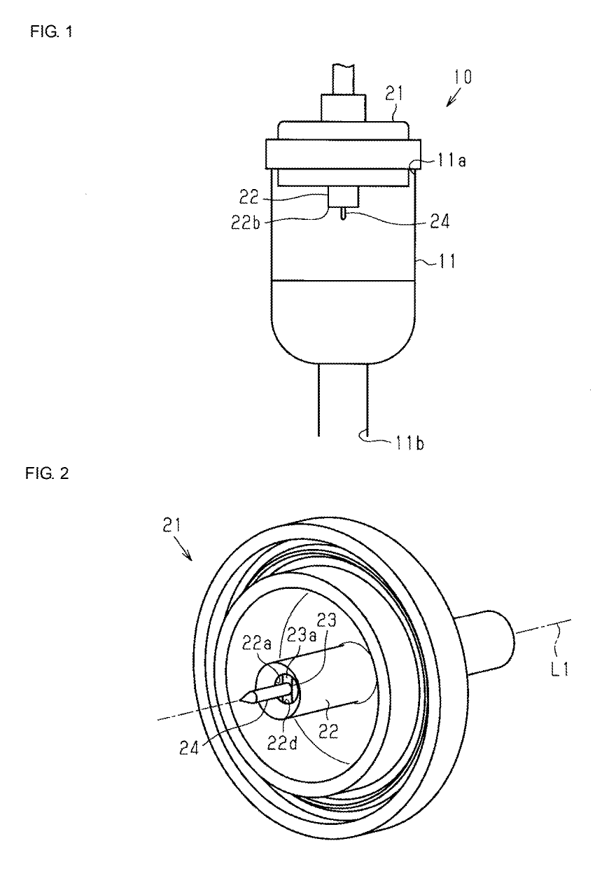

As illustrated in FIG. 1, a drip cylinder 10 according to the present embodiment includes a housing 11, and a cap 21 that is provided with a nozzle 22.

The housing 11 is made of, for example, a transparent resin material. The housing 11 is formed in a substantially cylindrical shape having open ends. The cap 21 is attached to an opening 11a of the housing 11, and a tube (not illustrated) is attached to another opening 11b of the housing 11.

As illustrated in FIG. 1, the cap 21 has a substantially disc-like shape and is attached to the upper opening 11a of the housing 11.

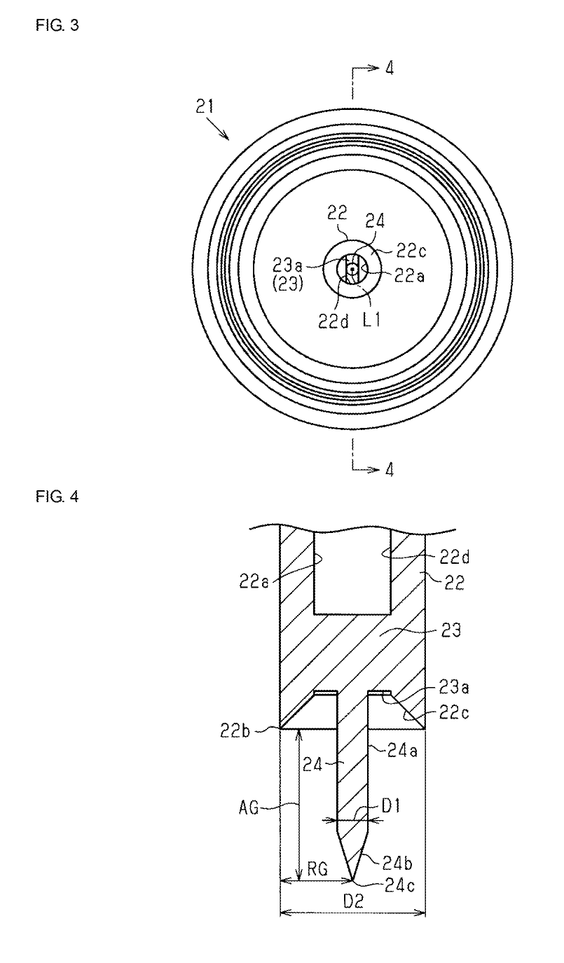

As illustrated in FIG. 2 and FIG. 3, the cap 21 is formed such that the nozzle 22 having an inflow hole 22a that is formed in the nozzle 22 and into which a liquid (a liquid medicine) can flow extends on a central axis L1 of the cap 21.

As illustrated in FIG. 1, an end portion 22b of the inflow hole 22a is formed so as to be located in the housing 11 in a state where the cap 21 is attached to the housing 11.

As illustrated in FIG. 4, the inflow hole 22a of the nozzle 22 is a substantially straight hole and has a substantially circular shape when viewed in a direction in which the hole extends through the nozzle 22. The inflow hole 22a is formed such that a portion thereof extends while having approximately the same diameter to an intermediate position and has an inverted tapered surface 22c that is formed such that the diameter of the inflow hole 22a is increased from the intermediate position to the end portion 22b. Thus, the diameter of the inflow hole 22a differs between the end portion 22b and the other portions of the inflow hole 22a.

As illustrated in FIG. 2 to FIG. 4, in the inflow hole 22a of the nozzle 22, an extended wall portion 23 that extends from an inner wall 22d of the inflow hole 22a is formed. The extended wall portion 23 is formed such that the end portions thereof connect or bridge portions of the inner wall 22d that are located at positions different from each other by 180 degrees in the circumferential direction of the inflow hole 22a.

As illustrated in FIG. 2 to FIG. 4, a needle-shaped portion 24 is integrally formed with a bottom surface 23a of the extended wall portion 23 so as to face a downstream side in a drip direction. Here, the needle-shaped portion 24 and the nozzle 22 are integrally formed by using the extended wall portion 23, the cap 21, and a resin material. In addition, the needle-shaped portion 24 is formed so as to extend from the bottom surface 23a of the extended wall portion 23 while being spaced apart from the inflow hole 22a.

As illustrated in FIG. 4, the needle-shaped portion 24 can include a columnar portion 24a that has a columnar shape and an end portion 24b that is tapered from an end portion of the columnar portion 24a. The end portion 24b is not open toward the housing 11. The columnar portion 24a and the end portion 24b of the needle-shaped portion 24 can each be formed as a solid portion, that is, a non-hollow portion.

The needle-shaped portion 24 is formed so as to project from the end portion 22b of the inflow hole 22a of the nozzle 22 toward the downstream side (the lower side) in the drip direction. The amount of projection of the needle-shaped portion 24 from the end portion 22b of the inflow hole 22a of the nozzle 22 can be within a range of larger than 0 mm to 5 mm or smaller. Note that these may be changed due to the relationship with, for example, the size of an opening at the end portion 22b of the inflow hole 22a of the nozzle 22.

In the case where a diameter D2 of the end portion 22b of the nozzle 22 is set to 4.0 mm, a diameter D1 of the needle-shaped portion 24 is set to 0.2 mm or larger and 3.0 mm or smaller. In other words, D1:D2 is set within a range of 1:20 to 3:4.

As illustrated in FIG. 4, a pointed tip 24c of the needle-shaped portion 24 that is the lowermost end of the needle-shaped portion 24 in a usage form of the drip cylinder 10 is spaced apart from the end portion 22b that is the lowermost edge of the inflow hole 22a in the axial direction of the needle-shaped portion 24 and in the radial direction of the needle-shaped portion 24. Thus, the end portion 22b of the inflow hole 22a and the pointed tip 24c of the needle-shaped portion 24 form an axial-direction gap AG (i.e., the projection amount) and a radial-direction gap RG therebetween. The axial-direction gap AG and the radial-direction gap RG contribute to reduction of the satellite droplets.

According to the present embodiment, which has been described above, the following advantageous effects can be obtained.

(1) The end portion 24b of the needle-shaped portion 24 is positioned further downstream than the end portion 22b of the inflow hole 22a of the nozzle 22 in the drip direction. Here, in a nozzle having a configuration of the related art, when a liquid that has collected in an end portion of an inflow hole of the nozzle is separated from the end portion of the inflow hole, the liquid tries to return into the nozzle (the inflow hole) by the surface tension of the liquid on the nozzle (the inflow hole) side. However, part of the liquid cannot completely return into the nozzle (the inflow hole) and separates as satellite droplets from the end portion of the inflow hole. In contrast, in the drip cylinder 10 according to the present embodiment, as described above, the needle-shaped portion 24 is positioned further downstream in the drip direction than the end portion 22b of the inflow hole 22a of the nozzle 22 where satellite droplets are likely to be generated, so that the liquid may easily return into the nozzle 22 through the needle-shaped portion 24 by the surface tension of the liquid. Therefore, generation of the satellite droplets can be suppressed.

(2) In addition, as described above, the needle-shaped portion 24 is positioned further downstream than the end portion 22b of the inflow hole 22a of the nozzle 22, where satellite droplets are likely to be generated, in the drip direction, so that the probability of the liquid spreading out into the end portion 22b of the nozzle 22 can be reduced, and the volume of the main droplets can be made further uniform.

(3) By providing the needle-shaped portion 24 at the center of the inflow hole 22a, the liquid that has collected in the end portion 22b of the inflow hole 22a and the needle-shaped portion 24 are likely to come into contact with each other with higher certainty, and thus, the liquid may easily return into the nozzle 22 with higher certainty through the needle-shaped portion 24 by the surface tension of the liquid.

(4) The ratio of the diameter D1 of the columnar portion to the diameter D2 of the inflow hole is set such that D1:D2 is within the range of 1:20 to 3:4, so that the flow rate of the liquid that is supplied from the nozzle can be stabilized while generation of satellite droplets is suppressed.

(5) Since the needle-shaped portion 24 and the nozzle 22 are integrally formed by using the resin material, an increase in the number of components can be suppressed.

(Modification)

Note that the above-described embodiment can be implemented in the following aspects that are obtained by suitably changing the embodiment.

In the above-described embodiment, although a configuration in which the inflow hole 22a of the nozzle 22 has the inverted tapered surface 22c is employed, the present disclosure is not limited to this configuration.

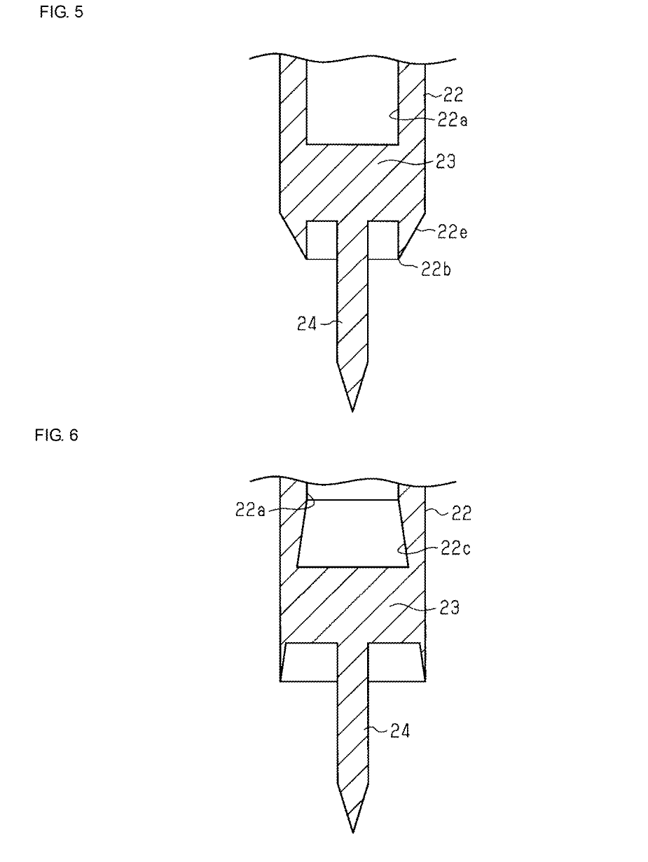

As illustrated in FIG. 5, the inner diameter of the inflow hole 22a may be approximately constant. In addition, in FIG. 5, a tapered surface 22e is formed such that the outer diameter of the nozzle 22 is decreased.

As illustrated in FIG. 6, the extended wall portion 23 may be formed at an intermediate position on the inverted tapered surface 22c.

In the above-described embodiment, although the extended wall portion 23 is formed such that the end portions thereof connect or bridge the portions of the inner wall 22d that are located at the positions different from each other by 180 degrees in the circumferential direction of the inflow hole 22a, the present disclosure is not limited to this configuration. For example, the extended wall portion 23 may be formed so as to extend from portions of the inner wall 22d that are located at positions different from one another by 120 degrees in the circumferential direction of the inflow hole 22a toward the center of the inflow hole 22a.

The extended wall portion 23 can have a hydrodynamic shape. For example, the extended wall portion 23 may be chamfered so as to have no corner. The extended wall portion 23 can have a streamline shape (a curved outer surface), and the extended wall portion 23 can have a wing shape. By employing the extended wall portion 23 having a hydrodynamic shape, an increase in the flow path resistance in the inflow hole 22a can be suppressed, and/or the flow of the liquid in the inflow hole 22a can be made uniform or rectified, so that the sizes of the liquid droplets are stabilized. Although an unintentional can take place in which liquid droplets are generated on only one side of the nozzle depending on the usage state of the drip cylinder, the extended wall portion 23 having a hydrodynamic shape is advantageous for suppressing occurrence of such an unintentional situation.

In the above-described embodiment, although a configuration in which the needle-shaped portion 24 projects from the inflow hole 22a of the nozzle 22 is employed, a configuration in which the needle-shaped portion 24 is positioned further downstream than the inflow hole 22a in the drip direction by holding (supporting) the needle-shaped portion 24 outside the inflow hole 22a may be employed.

In the above-described embodiment, although a configuration in which the needle-shaped portion 24 is provided at the center of the inflow hole 22a of the nozzle 22, the present disclosure is not limited to this configuration, and a configuration in which the needle-shaped portion 24 is provided at a position that is offset with respect to the center of the inflow hole 22a may be employed.

In the above-described embodiment, although the shape of the inflow hole 22a when viewed in the direction in which the inflow hole 22a extends through the nozzle 22 is a circular shape, the shape of the inflow hole 22a in this direction may be an elliptical shape or a polygonal shape.

In the above-described embodiment, although a configuration in which the columnar portion 24a, which is included in the needle-shaped portion 24, has a substantially columnar shape and in which the end portion 24b has a substantially conical shape is employed, the present disclosure is not limited to this configuration.

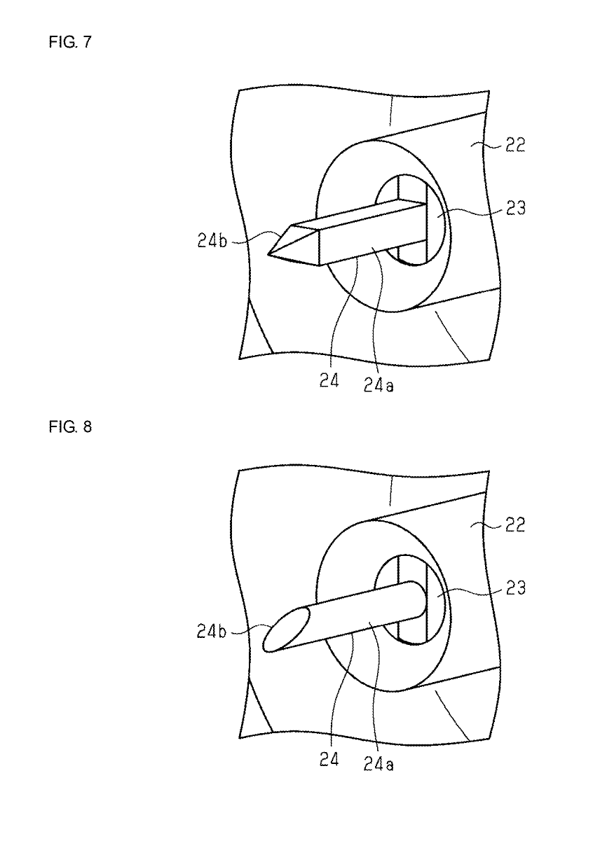

As illustrated in FIG. 7, a configuration in which the columnar portion 24a has a substantially square columnar shape and in which the end portion 24b has a substantially square pyramid-like shape may be employed. Alternatively, the columnar portion 24a may have a polygonal columnar shape other than a square columnar shape. The end portion 24b may have a polygonal pyramid-like shape other than a square pyramid-like shape.

Alternatively, as illustrated in FIG. 8, the end portion 24b having a shape like a cylinder that is obliquely cut may be employed.

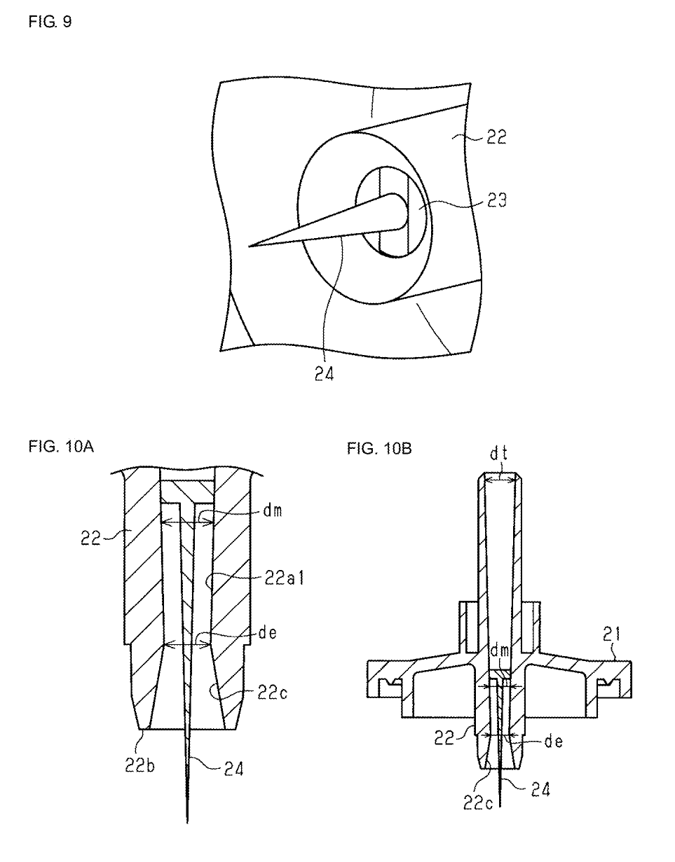

In the above-described embodiment although the needle-shaped portion 24 is formed of the columnar portion 24a and the end portion 24b that is tapered, as illustrated in FIG. 9, the needle-shaped portion 24 whose overall shape is gradually tapered may be employed. In addition, the end portion 24b is not limited to a so-called sharp corner and may have a shape that is tapered with a curved surface shape.

The inner diameter of the inflow hole 22a may be gradually decreased in the drip direction. For example, in the case illustrated in FIG. 10A, the diameter of a portion 22a1 of the inflow hole 22a that extends from the extended wall portion 23 to the inverted tapered surface 22c is gradually decreased in the drip direction. A minimum inner diameter de of the inverted tapered surface 22c is smaller than an inner diameter dm of the inflow hole 22a at the extended wall portion 23 (de<dm). In the case illustrated in FIG. 10B, the diameter of the entire inflow hole 22a extending from an upper opening of the inflow hole 22a to the inverted tapered surface 22c is gradually decreased in the drip direction. The minimum inner diameter de of the inverted tapered surface 22c is smaller than the inner diameter dm of the inflow hole 22a at the extended wall portion 23 and also smaller than an inner diameter dt of the inflow hole 22a at the upper opening of the inflow hole 22a, and the inner diameter dm of the inflow hole 22a is smaller than the inner diameter dt of the inflow hole 22a at the upper opening (de<dm<dt). In the case where the nozzle 22 does not have the inverted tapered surface 22c, the diameter of a portion of the inflow hole 22a that extends from the upper end opening of the inflow hole 22a or from the extended wall portion 23 to the end portion 22b may be gradually decreased in the drip direction. With this configuration, the flow of the liquid in the entire inflow hole 22a or in a portion of the inflow hole 22a extending from the extended wall portion 23 to the end portion 22b is made uniform or rectified, and the sizes of the liquid droplets are stabilized. For example, occurrence of a situation in which liquid droplets are generated on only one side of the needle-shaped portion can be suppressed.

In the above-described embodiment and the modifications, although the needle-shaped portion 24 and the nozzle 22 are made of the same material and integrally formed into one product, the present disclosure is not limited to this configuration, and the needle-shaped portion 24 and the nozzle 22 may be made of different materials. For example, there is a method in which the nozzle 22 is made of a resin material and in which the needle-shaped portion 24 is made of a metal material.

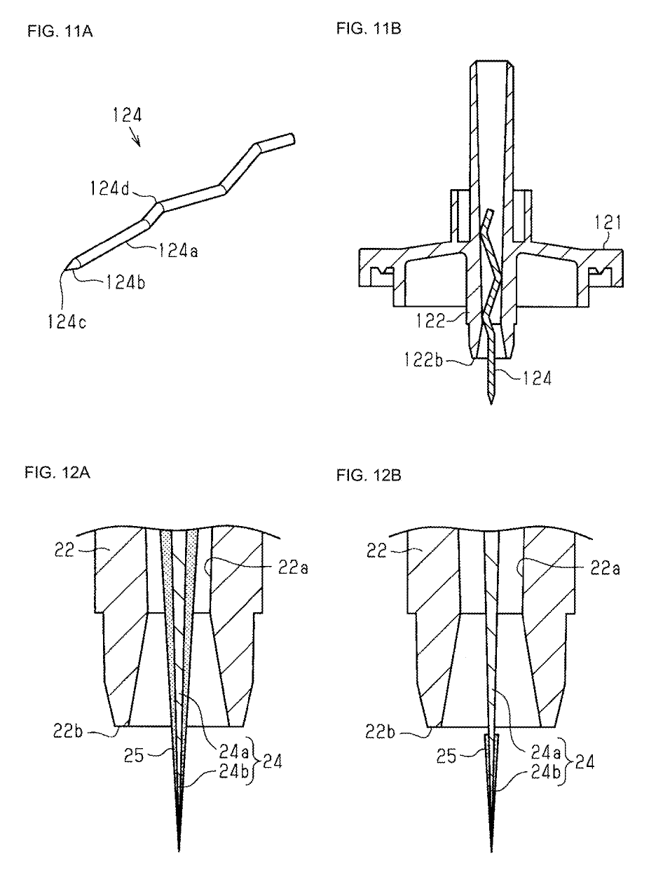

A modification in which a needle-shaped portion and a nozzle are made of different materials will be described with reference to FIGS. 11A and 11B. A needle-shaped portion 124 is an individual member made of a material such as a metal that has elasticity. The needle-shaped portion 124 can include a columnar portion 124a having a linear shape, an end portion 124b having a pointed tip 124c, and a spring-shaped portion 124d that is a base portion. In the case illustrated in FIGS. 11A and 11B, although the spring-shaped portion 124d has a zigzag shape, the shape of the spring-shaped portion 124d is not particularly limited, and for example, the spring-shaped portion 124d may have a different spring shape such as the shape of a banana plug or a spiral or helical shape.

A nozzle 122 is made of a synthetic resin having water repellency. As illustrated in FIG. 11B, for example, the nozzle 122 is integrally formed with a cap 121 as a portion of the cap 121. The difference between the nozzle 122 and the nozzle 22 according to the above-described embodiment is that the nozzle 122 does not include the needle-shaped portion 24 according to the above-described embodiment.

The spring-shaped portion 124d of the needle-shaped portion 124 is inserted in an inflow hole 122a of the nozzle 122 and is configured to be elastically compressed in a radial direction in the inflow hole 122a of the nozzle 122. The needle-shaped portion 124 is fixedly mounted on the nozzle 122 by the elastic restoring force of the spring-shaped portion 124d. The end portion 124b of the needle-shaped portion 124, which includes the pointed tip 124c, projects in the drip direction from an end portion 122b that is the lowermost edge of the inflow hole 122a of the nozzle 122.

The needle-shaped portion 124, which is an individual member, can provide the following advantageous effects.

The needle-shaped portion 124 can be incorporated into an existing cylindrical nozzle that does not include a needle-shaped portion. The needle-shaped portion 124 and the nozzle 122 (the cap 121) can be manufactured through different processes. In general, in order to stabilize the sizes of liquid droplets, it is desirable that a nozzle of a drip cylinder be made of a water-repellent material so as to make the liquid droplets less likely to spread up along the outer surface of the nozzle. In contrast, in order to suppress generation of satellite droplets, it is desirable that a needle-shaped portion be made of a hydrophilic material so as to cause the liquid droplets to spread out onto a surface of a needle-shaped portion. In order to satisfy these contradictory requirements, in the case where the needle-shaped portion 24 and the nozzle 22 are integrally formed of the same material as in the above-described embodiment, the needle-shaped portion 24 is formed into an extremely thin shape in order to reduce the water repellent effect of the needle-shaped portion 24. Integral molding process of the nozzle 22 including the thin needle-shaped portion 24 has a high degree of difficulty. For example, there is a case where the manufacturing costs of a metal mold for integral molding is high. Regarding this, in the case where the needle-shaped portion 124 is an individual member that is different from the nozzle 122 as in the modification illustrated in FIGS. 11A and 11B, the needle-shaped portion 124, which is very thin, can be solely manufactured, and this makes it easier to perform the forming process of the nozzle 122 that does not include a needle-shaped portion. The needle-shaped portion 124 and the nozzle 122 can be easily made of different materials, and for example, the nozzle 122 and the needle-shaped portion 124 can be respectively made of a water-repellent material and a hydrophilic material. Thus, the effect of stabilizing the sizes of liquid droplets and the effect of suppressing generation of satellite droplets can be both achieved at higher level and at low cost.

As illustrated in FIG. 12A, the needle-shaped portion 24 may include a hydrophilic surface layer 25 on the outermost surface thereof. For example, the hydrophilic surface layer 25 can be formed by surface modification, such as coating, a plasma treatment, an UV treatment, or a frame treatment, that improves hydrophilicity. The hydrophilic surface layer 25 enables liquid droplets to easily spread out onto the surface of the needle-shaped portion 24, and the effect of suppressing satellite droplets that is obtained by the needle-shaped portion 24 is stabilized and/or improved. A desired effect of suppressing satellite droplets can be obtained by the hydrophilic surface layer 25 without necessarily forming the needle-shaped portion 24 into an extremely thin shape. Thus, the hydrophilic surface layer 25 is particularly advantageous in the case where the needle-shaped portion 24 is integrally formed with the nozzle 22. Note that, as illustrated in FIG. 12B, the hydrophilic surface layer 25 may be locally provided on a portion of the needle-shaped portion 24 including the end portion 24b.

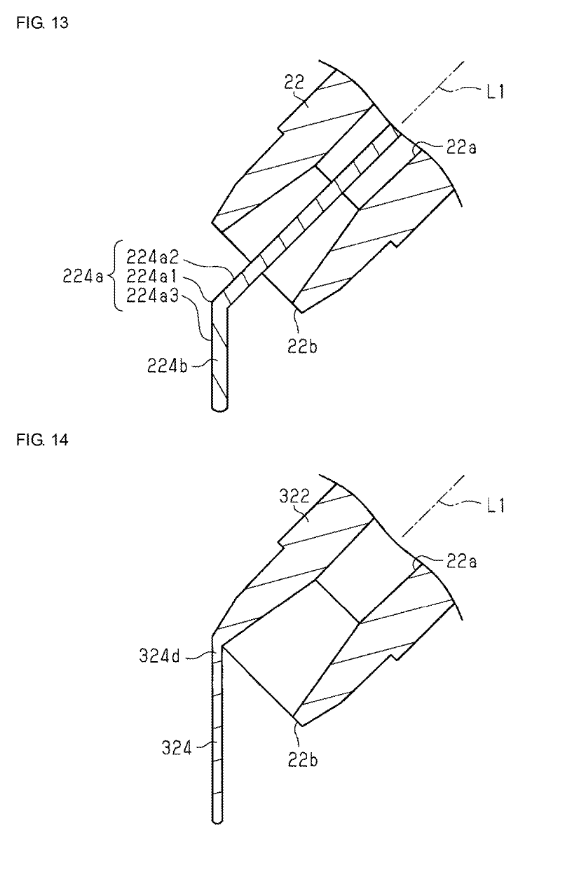

In the above-described embodiment, the entire needle-shaped portion 24 has a linear shape, and the entire needle-shaped portion 24 is disposed so as to be parallel to or so as to be on the central axis L1 of the cap 21 or the nozzle 22. However, in the modification illustrated in FIG. 13, a needle-shaped portion 224 includes a columnar portion 224a that includes a bent portion 224a1 and an end portion 224b that includes a pointed tip 224c. The bent portion 224a1 is provided between a linear upper portion 224a2 of the columnar portion 224a and a linear lower portion 224a3 of the columnar portion 224a. The linear lower portion 224a3 is bent at the bent portion 224a1 with respect to the linear upper portion 224a2. The linear upper portion 224a2 of the columnar portion 224a is disposed so as to be parallel to or so as to be on the central axis L1 of the cap 21 or the nozzle 22. In contrast, the end portion 224b (and the lower portion 224a3 of the columnar portion 224a) is not parallel to the central axis L1. The needle-shaped portion 224 projects from the end portion 22b of the inflow hole 22a of the nozzle 22 in the drip direction. Also, with this configuration, the effect of suppressing generation of satellite droplets can be obtained.

In order to reduce the probability of liquid droplets adhering to the inner wall of the housing 11, the dripping cylinder 10 may sometimes be used such that the axis of the housing 11 is inclined with respect to the direction of gravity. As a result of the dripping cylinder 10 including the needle-shaped portion 224 being used in an inclined state, the probability of liquid droplets adhering to the inner wall of the housing 11 is further reduced.

In the above-described embodiment, the base portion of the needle-shaped portion 24 is supported by the inner wall 22d of the inflow hole 22a of the nozzle 22. However, the needle-shaped portion is not necessarily supported by the inner wall 22d of the inflow hole 22a. For example, in the case illustrated in FIG. 14, a base portion 324d of a needle-shaped portion 324 is directly connected to a portion of an end portion 22b of an inflow hole 22a of a nozzle 322. The needle-shaped portion 324 projects from the end portion 22b of the inflow hole 22a of the nozzle 322 in the drip direction. The nozzle 322 is disposed so as to be parallel to or so as to be on the central axis L1 of the cap 21. In contrast, the entire needle-shaped portion 324 is not parallel to the central axis L1. The needle-shaped portion 324 is bent at the base portion 324d with respect to the nozzle 322. The needle-shaped portion 324 projects from the end portion 22b of the inflow hole 22a of the nozzle 322 in the drip direction. Also, with this configuration, the effect of suppressing generation of satellite droplets can be obtained. As a result of the dripping cylinder 10 including the needle-shaped portion 324 being used in an inclined state, the probability of liquid droplets adhering to the inner wall of the housing 11 is further reduced.

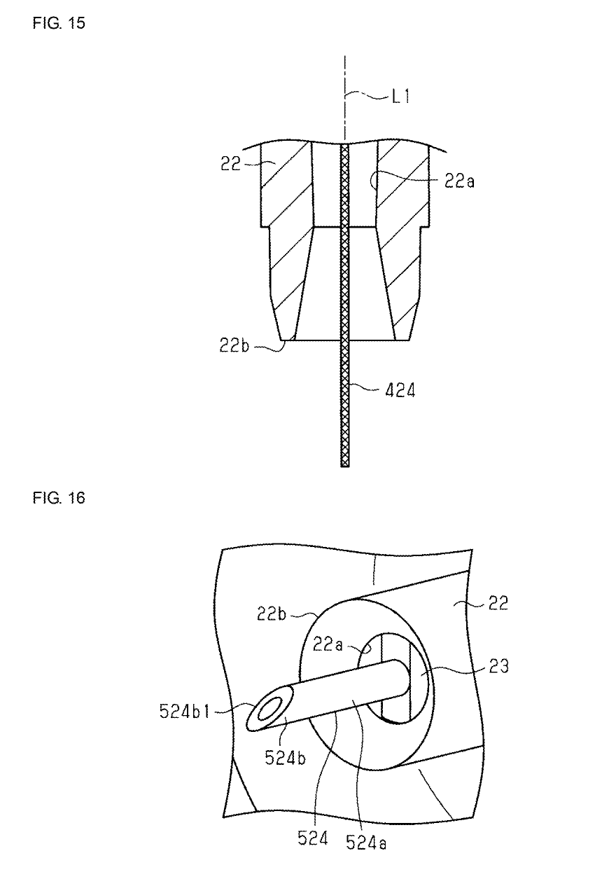

In the above-described embodiment, the columnar portion 24a and/or the end portion 24b of the needle-shaped portion 24 is configured to maintain its initial shape. However, the needle-shaped portion may be formed so as to be deformable at the columnar shape and/or the end portion. For example, in the modification illustrated in FIG. 15, a needle-shaped portion 424 is formed as a flexible string. The needle-shaped portion 424 projects from the end portion 22b of the inflow hole 22a of the nozzle 22 in the drip direction. Also, with this configuration, the effect of suppressing generation of satellite droplets can be obtained. As a result of the dripping cylinder 10 including the needle-shaped portion 424 being used in an inclined state, the probability of liquid droplets adhering to the inner wall of the housing 11 is further reduced.

In the above-described embodiment and the modifications, the needle-shaped portion 24 is formed as a solid member. However, the needle-shaped portion may be formed as a hollow member. For example, in the modification illustrated in FIG. 16, a hollow needle-shaped portion 524 includes a cylindrical columnar shape 524a and an end portion 524b that has an end opening. The end portion 524b has an annular end surface 524b1 obliquely crossing the axis of the columnar shape 524a, and the end opening is defined by the annular end surface 524b1. The needle-shaped portion 524 projects from the end portion 22b of the inflow hole 22a of the nozzle 22 in the drip direction. Also, with this configuration, the effect of suppressing generation of satellite droplets can be obtained.

The above-described embodiment and the above-described modifications may be suitably combined.

### REFERENCE SIGNS LIST

10 drip cylinder
11 housing
21 cap
22 nozzle
22a inflow hole
24 needle-shaped portion
24a columnar portion
24b end portion (end)
D1, D2 diameter

1. A drip cylinder comprising:
a housing;
a nozzle that has an inflow hole which allows a liquid to flow into the housing and that causes the liquid to drip from an end of the inflow hole; and
a needle-shaped portion in which at least an end portion of the needle-shaped portion is positioned downstream of the end of the inflow hole of the nozzle in a drip direction.

2. The drip cylinder according to claim 1,
wherein the needle-shaped portion is spaced apart from the inflow hole.

3. The drip cylinder according to claim 1,
wherein the needle-shaped portion is disposed at a center of the inflow hole when an opening surface of the inflow hole is viewed from the front.

4. The drip cylinder according to claim 1,
wherein the needle-shaped portion includes a columnar portion and an end portion.

5. The drip cylinder according to claim 1,
wherein the needle-shaped portion includes a cylindrical columnar portion,
wherein the nozzle has a circular inflow hole, and
wherein the ratio of a diameter D1 of the cylindrical columnar portion to a diameter D2 of the circular inflow hole is set such that D1:D2 is within a range of 1:20 to 3:4.

6. The drip cylinder according to claim 2,
wherein the needle-shaped portion is disposed at a center of the inflow hole when an opening surface of the inflow hole is viewed from the front.

7. The drip cylinder according to claim 2,
wherein the needle-shaped portion includes a columnar portion and an end portion.

8. The drip cylinder according to claim 3,
wherein the needle-shaped portion includes a columnar portion and an end portion.

9. The drip cylinder according to claim 2,
wherein the needle-shaped portion includes a cylindrical columnar portion,
wherein the nozzle has a circular inflow hole, and
wherein the ratio of a diameter D1 of the cylindrical columnar portion to a diameter D2 of the circular inflow hole is set such that D1:D2 is within a range of 1:20 to 3:4.

10. The drip cylinder according to claim 3,
wherein the needle-shaped portion includes a cylindrical columnar portion,
wherein the nozzle has a circular inflow hole, and
wherein the ratio of a diameter D1 of the cylindrical columnar portion to a diameter D2 of the circular inflow hole is set such that D1:D2 is within a range of 1:20 to 3:4.

11. The drip cylinder according to claim 4,
wherein the needle-shaped portion includes a cylindrical columnar portion,
wherein the nozzle has a circular inflow hole, and
wherein the ratio of a diameter D1 of the cylindrical columnar portion to a diameter D2 of the circular inflow hole is set such that D1:D2 is within a range of 1:20 to 3:4.
